(19) United States
(12) Patent Application Publication
(10) Pub. No.: US 2007/0095698 A1
(43) Pub. Date: May 3, 2007

(54) Apparatus for storing biological prostheses

(76) Inventor: Ronald E. Cambron, Laguna Hills, CA (US)

Correspondence Address: Vista IP Law Group LLP, 2040 MAIN STREET, 9TH FLOOR, IRVINE, CA, 92614, US

(21) Appl. No.: 11/250,501

(22) Filed: Oct. 14, 2005

(51) Int. Cl. A61B 19/02; B65D 17/34; B65D 51/20; B65B 51/10; B65B 7/28

(52) U.S. Cl. 206/438; 220/359.2; 220/258.2; 53/485; 53/477

(57) ABSTRACT

An apparatus for storing a bioprosthetic device includes a container for receiving the bioprosthetic device within an opening in the container. The storage apparatus includes an induction seal having a foil layer and a heat seal layer for sealing the opening of the container. A pull tab is located on the induction seal and includes an aperture therein that facilitates bonding the induction seal to the container and/or otherwise sealing the container. A substantially hermetic seal is created by the induction seal. The apparatus may be opened with relative ease by pulling on the pull tab. The apparatus also reduces the risk of spillage of sterilant solution upon opening.

### FIELD OF THE INVENTION

The present invention relates generally to apparatus and methods for storing bioprostheses, such as bioprosthetic heart valves.

### BACKGROUND

Prosthetic heart valves can replace defective human valves in patients. Prosthetic heart valves may be formed from biological tissues and/or mechanical components. Typically, prosthetic heart valves are assembled and placed into a storage container, such as a jar. The jar is sealed until opened by the physician or other health care professional in the operating room. Typically, screw lids (with and without a liner) have been used to seal the prosthetic heart valve within the jar.

The screw lid design has, however, a number of drawbacks. First, to ensure that a good seal is formed between the lid and jar, the lids are tightened with a high degree of torque. This can make the lid hard to remove from the jar. In addition, the tight seal formed between the lid and jar often causes spillage during opening. When this occurs, the storage solution within the jar (such as glutaraldehyde sterilant solution) may spill out in the operating room and even contact operating room personnel.

Thus, storage devices for bioprosthetic devices that overcome the problems associated with conventional storage jars would be useful. The device preferably is able to form a good seal while at the same time is relatively easy to open. The device would also minimize or mitigate the risk of spillage during opening.

### SUMMARY OF THE INVENTION

The present invention is directed to an apparatus for storing a bioprosthetic device, such as heart valves, and/or to methods for making, assembling, and/or using them.

In accordance with one embodiment, the apparatus includes a container for receiving a bioprosthetic device within an opening formed in the container. The apparatus further includes an induction seal for sealing the opening of the container. The induction seal includes a foil layer and a heat seal layer for sealing the opening. The induction seal includes a pull tab having an aperture therein.

In one embodiment, the pull tab and the aperture therein have a triangular shape. For example, the pull tab and aperture may be oriented such that a vertex of the aperture is disposed adjacent to a vertex formed in the pull tab.

In accordance with another embodiment, a method for storing a bioprosthetic device includes providing a container, the container including an opening formed therein. An induction seal is provided for sealing the opening of the container. The induction seal includes a foil layer and a heat seal layer for inductively sealing the opening of the container. The induction seal further includes a pull tab having an aperture formed therein. The induction seal is sealed over the opening formed in the container with a heat-induction generator. The method may be used to store a bioprosthetic device, such as a heart valve.

In accordance with still another embodiment, a product is produced by the process of providing a container including an opening formed therein. A bioprosthetic device is inserted into the container via the opening. An induction seal is provided for sealing the opening of the container, the induction seal includes a foil layer and a heat seal layer, the induction seal further includes a pull tab with an aperture therein. The induction seal is then sealed over the opening formed in the container with a heat-induction generator.

Other aspects and features of the invention will become apparent from consideration of the following description taken in conjunction with the accompanying drawings.

### BRIEF DESCRIPTION OF THE DRAWINGS

FIG. 1 is an exploded perspective view of an apparatus for storing a bioprosthetic device.

FIG. 2A is a plan view of the induction seal, according to one embodiment.

FIG. 2B is a cross-sectional view of the induction seal shown in FIG. 2A.

FIG. 3 is a perspective view of a heat induction generator being used to form a seal between the induction seal and the container of FIG. 1.

FIG. 4 is a perspective view of a sealed apparatus for storing a bioprosthetic device. The lid is shown removed to show the interface between the induction seal and the container.

### DETAILED DESCRIPTION OF THE PREFERRED EMBODIMENTS

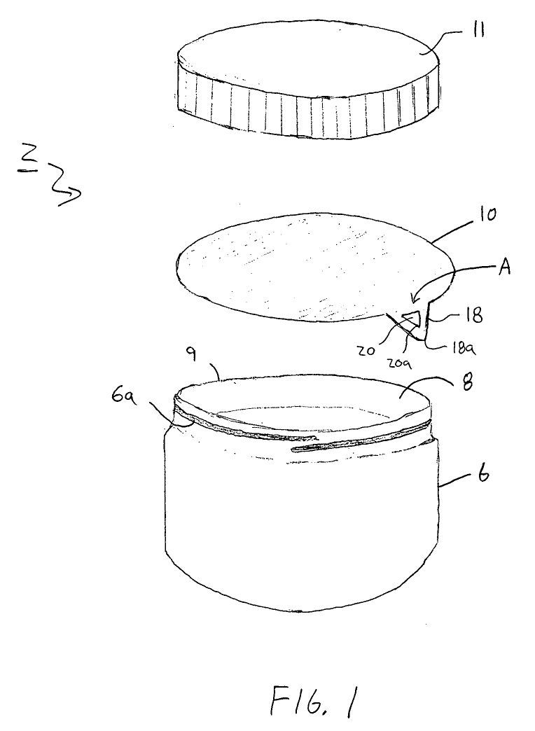

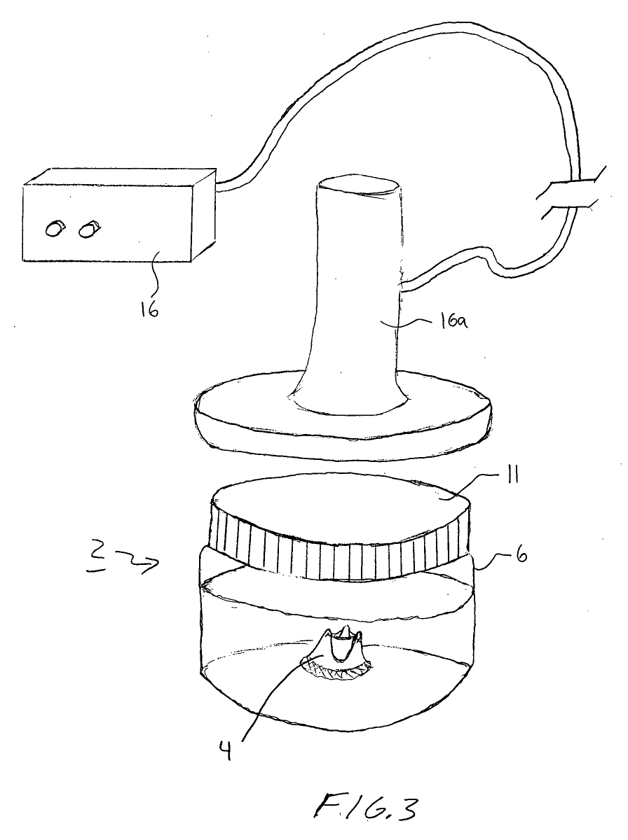

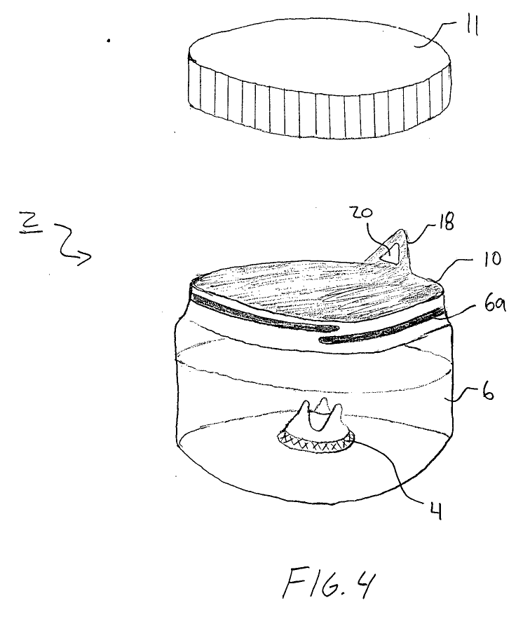

Turning to the drawings, FIGS. 1, 3, and 4 show an apparatus 2 for storing a bioprosthetic device 4 (as shown in FIGS. 3 and 4) such as, for instance, a bioprosthetic heart valve. The apparatus 2 includes a container 6 having an opening 8 defined by a lip 9 for receiving the bioprosthetic device 4. The container 6 may take the form of an open-ended receptacle such as, a threaded plastic jar (e.g., a polyethylene terephtalate (PET) based jar). The apparatus 2 further includes an induction seal 10 for sealing the opening 8 of the container 6. The induction seal 10 is able to create a hermetically sealed environment within the interior portion of the container 6. The apparatus 2 may include a cap or lid 11 that engages with corresponding threads 6a of the container 6. The interior of the lid 11 may contain a backing material (e.g., Saint-Gobain F-1299-2 liner).

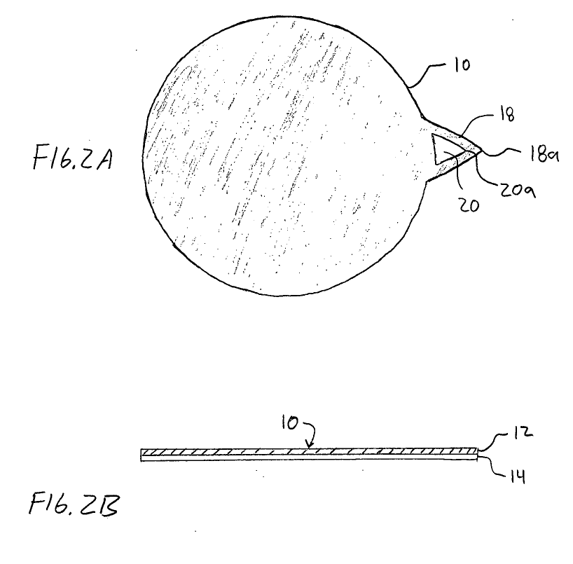

Turning to FIGS. 2A and 2B, the induction seal 10 may include a foil layer 12 and a heat seal layer 14. The foil layer 12 is formed from an electrically conductive material capable of heating in response to the application of radiofrequency (RF) energy from a heat-induction generator 16 (shown in FIG. 3 and described in more detail below). The heat seal layer 14 is generally formed from a heat-sensitive adhesive material that is used to bond the induction seal 10 to the container 6 in response to RF energy supplied by the heat-induction generator 16. The heat seal layer 14 may be formed on the entire surface of the induction seal 10 or, alternatively, just around the periphery of the induction seal 10 where the induction seal 10 contacts the container 6.

Referring to FIGS. 1, 2A, and 4, the induction seal 10 includes a pull tab 18. The pull tab 18 extends outwardly from the outer periphery of the induction seal 10. To break the seal formed between the induction seal 10 and the container, the pull tab 18 is gripped by the user and pulled in an upward fashion to open the apparatus 2. The pull tab 18 is formed with an aperture 20 therein to direct the heat energy created by the heat-induction generator 16 toward the interface of the induction seal 10 and the container 6 located radially inward from the pull tab 18 (identified by arrow A in FIG. 1).

It has been discovered that the absence of the aperture 20 in the pull tab 18 causes poor seal formation in the region identified by arrow A in FIG. 1. This is likely due to the fact that, without the aperture 20, the inducted heat energy concentrates in the portions of the pull tab 18 that lie outside the periphery of interface between the induction seal 10 and the container 6. Consequently, the heat seal layer 14 located in the region identified by arrow A undergoes incomplete heating, thereby causing poor sealing in this region.

The aperture 20 advantageously focuses the heat induction energy into the region identified by arrow A in FIG. 1. The focusing or redirection of energy in this region causes a good seal to form between the induction seal 10 and the container 6. If the aperture 20 were not used in the pull tab 18, poor sealing may result between the induction seal 10 and the container 6, thereby jeopardizing the hermetically sealed environment therein.

In one embodiment, shown in FIGS. 1, 2A, and 4, the pull tab 18 has a triangular shape that terminates in an apex or vertex 18a away from the main body of the induction seal 10. As best seen in FIG. 2A, the aperture 20 has a triangular shape with an apex or vertex 20a disposed adjacent to the vertex 18a formed in the pull tab 18, i.e., the aperture 20 is located concentrically within the pull tab 18. In one embodiment, the triangular aperture 20 is completely located within the pull tab 18.

While a triangular shaped pull tab 18 and aperture 20 are shown in FIGS. 1, 2A, and 4, it should be understood that other geometric-shaped pull tabs 18 and/or apertures 20 may be provided. For example, the aperture 20 may be circular, square, rectangular, or polygonal within a triangular or other shaped pull tab 18. Thus, the pull tab 18 may define a surface area surrounding the aperture 20. The size of open area defined by the aperture 20 may be greater than the remaining surface area of the pull tab 18 or, alternatively, the open area of the aperture 20 may be less than the surface area of the pull tab 18. By minimizing the surface area of the pull tab 18, the energy from induction heating may be focused on the heat seal layer, rather than being dissipated out onto the pull tab 18.

For packaging a bioprosthetic device 4, such as a heart valve, the container 6, bioprosthetic device 4, lid 11, and induction seal 10 may be placed in a clean room environment. The clean room environment may contain a laminar flow hood or other working area (not shown) used to aseptically transfer the bioprosthetic device 4 from a separate aseptic container (not shown). The container 6, lid 11, and induction seal 10 may be sterilized by wiping exposed surfaces with an antimicrobial agent, for example, a solution of seventy percent (70%) isopropyl alcohol (IPA).

The container 6 is then filled with terminal sterilant solution prior to transfer. For example, a terminal sterilant solution may be used, such as that disclosed in co-pending U.S. patent application Ser. No. 11/032,923, the entire disclosure of which is expressly incorporated by reference herein. Enough terminal sterilant solution may be added to completely cover the bioprosthetic device 4.

The bioprosthetic device 4 is then aseptically transferred into the container 6, for example, using autoclaved forceps. An induction seal 10 may then be prepared for insertion into the lid 11 of the apparatus 2. The pull tab 18 is partially folded (about 90°) to permit the placement of the induction seal 10 inside the lid 10. A similarly sized diameter template may be used to assist in folding the pull tab 18. The template may include, for example, another induction seal 10 of the same size. In the case of Selig S70 FS 3-91 die-cut induction seals 10, the fold is made toward the silver side of the induction seal 10. The induction seal 10 is then placed inside the lid 11 with the silver side exposed. The induction seal 10 is oriented such that the pull tab 18 is located about one tab width to the right of the ending point of the inner lid thread 6a.

The lid 11 (with the induction seal 10 contained therein) is then positioned over the opening 8 of the container. The lid 11 is then gently screwed until rotation of the lid 11 stops. The container 6 (with screwed lid 11) is then transferred to a torque tester riser block assembly (not shown) to tighten the lid 11. The lid is tightened to around twenty two inch-pounds (22 in-lbs) of torque (+/−2 in-lbs).

After tightening, the container 6 is transferred to the heat-induction generator 16 for sealing (as seen in FIG. 3). For example, the heat-induction generator 16 may include a RELCO ICS-1H hand-held heat-induction generator, available from Relco UK, Ltd. The power setting may be set to 4.5+/−1.0 with a cycle duration of 3.4. The hand-held sealer portion 16a of the heat-induction generator 16 is placed on top of the container 6.

The heat-induction generator 16 is triggered via a button or other trigger (not shown) and held in place until a beep (or other indicator) indicates that the hand-held sealer portion 16a may be removed. As a result, the heat seal layer 14 of the induction seal 10 may be at least partially melted or otherwise bonded to the lip 9 of the container 6, thereby substantially sealing the interior of the container 6 from the surrounding environment.

After sealing has been accomplished, the containers 6 may be subject to vacuum leak testing and sterilization. The container 6 now contains the bioprosthetic device 4, which may be stored for later use.

FIG. 4 illustrates a bioprosthetic device 4 stored inside a sealed container 6 with the lid 11 removed. To open the assembly, a user first unscrews the lid 11 from the container 6. The user next grabs the pull tab 18 and pulls in an upward direction to release the induction seal 10 from the opening 8 of the container 6. The pull tab 18 advantageously permits easy removal of the induction seal 10 to access to the interior of the container 6. In addition, the ease of removal of the induction seal 10 means that spillage of the terminal sterilant solution is avoided. The bioprosthetic device 4 may then be implanted within a patient or otherwise used to treat a patient.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

What is claimed is:

1. An apparatus for storing a bioprosthetic device comprising;
a container for receiving a bioprosthetic device within an opening in the container;
an induction seal for sealing the opening of the container, the induction seal including a foil layer and a heat seal layer, the induction seal having a pull tab with an aperture therein.

2. The apparatus of claim 1, wherein the pull tab has a triangular shape.

3. The apparatus of claim 2, wherein the aperture has a triangular shape.

4. The apparatus of claim 3, wherein the aperture includes a vertex disposed adjacent to a vertex formed in the pull tab.

5. The apparatus of claim 1, wherein the aperture is completely located within the pull tab.

6. The apparatus of claim 1, wherein the aperture defines an open area that is greater than a surface area of the pull tab.

7. The apparatus of claim 1, wherein the aperture defines an open area that is less than a surface area of the pull tab.

8. The apparatus of claim 1, wherein the bioprosthetic device comprises a heart valve.

9. The apparatus of claim 8, further comprising a sterilant solution within the container.

10. A method for storing a bioprosthetic device within a container including an opening, comprising:
placing an induction seal over the opening, the induction seal including a foil layer and a heat seal layer, the induction seal having a pull tab with an aperture formed therein; and
sealing the induction seal over the opening with a heat-induction generator.

11. The method of claim 10, wherein the pull tab has a triangular shape.

12. The method of claim 11, wherein the aperture has a triangular shape.

13. The method of claim 12, wherein the aperture includes a vertex disposed adjacent to a vertex formed in the pull tab.

14. The method of claim 10, wherein the aperture is completely located within the pull tab.

15. The method of claim 10, further comprising placing a bioprosthetic device in the container before sealing the induction seal.

16. The method of claim 15, wherein the bioprosthetic device comprises a heart valve.

17. The method of claim 10, further comprising placing a sterilant solution in the container before sealing the induction seal.

18. The method of claim 10, wherein the induction seal is placed over the opening with the heat seal layer against a lip of the container defining the opening.

19. The method of claim 10, wherein the induction seal is placed in a lid before placing the induction seal over the opening.

20. The method claim 19, wherein the lid is threaded onto the container over the opening to place the induction seal over the opening.

21. A product produced by the process of:
providing a container including an opening therein;
inserting a bioprosthetic device into the container via the opening;
placing an induction seal over the opening, the induction seal including a foil layer and a heat seal layer, the induction seal having a pull tab with an aperture therein; and
sealing the induction seal over the opening with a heat-induction generator.
